(19) United States
(12) Patent Application Publication
(10) Pub. No.: US 2024/0374243 A1
(43) Pub. Date: Nov. 14, 2024

(54) ULTRASOUND IMAGING USING FOCUSING BEAMS FOR REDUCING MECHANICAL INDEX AND THERMAL INDEX

(71) Applicant: CLOUDSTREAM MEDICAL IMAGING, INC., Houston, TX (US)

(72) Inventors: Chengbin PENG, Houston, TX (US); Jun TANG, Houston, TX (US)

(21) Appl. No.: 18/686,864

(22) PCT Filed: Sep. 9, 2022
(86) PCT No.: PCT/US2022/043133

§ 371 Date: Feb. 26, 2024

Related U.S. Application Data
(60) Provisional application No. 63/243,325, filed on Sep. 13, 2021.

(51) Int. Cl. A61B 8/00

(52) U.S. Cl. CPC A61B 8/543; A61B 8/4483; A61B 8/461

(57) ABSTRACT

A system and method for acquiring and beamforming ultrasound data using focusing beams includes: providing an ultrasound transducer, the ultrasound transducer including a plurality of elements; transmitting ultrasound beams from the elements in such a way that sound waves arrive at a focal point at different times but within predetermined time differences, the differences being (i) small enough such that the in-sonification at the focal point is strong to overcome noises and attenuation of echo signals caused by tissue absorption, and (ii) large enough to avoid a constructive interference of the sound waves at the focal point; receiving raw RF ultrasound data using at least a subset of the elements for each beam; beamforming the RF ultrasound data to obtain an image; displaying the image or sending the image to a remote device. Ultrasound scanners using focusing beams can achieve excellent image resolution and signal-to-noise ratio, significantly reducing safety concerns.

This application claims priority to U.S. Provisional Patent Application No. 63/243,325, filed on Sep. 13, 2021, which is incorporated by reference for all purposes as if fully set forth herein.

### FIELD OF THE INVENTION

The present invention relates to ultrasound imaging using focusing beams for reducing mechanical index and thermal index, and more precisely, to a system and method of acquiring focusing beam data and generating high quality images.

### BACKGROUND OF THE INVENTION

Medical ultrasound imaging for diagnosis has advantages, such as reasonable cost, real-time imaging, portability, and its harmless effect, over computerized tomography (CT) and magnetic resonance imaging (MRI) [1-2]. However, the resolution of the ultrasound imaging system is usually lower than that of CT and MRI systems [3]. Ultrasound imaging technology is progressing towards high quality and sharp resolution, thanks to better data acquisition hardware and sophisticated processing software [4].

Commonly used ultrasound transducers include linear array transducers, curved array transducers, and phased array transducers. Ultrasound images of a linear array transducer have a rectangular shape. Since the linear array is normally used for precise imaging, its operating frequency is high. In contrast, the convex array is used to acquire a wide and deep ultrasound image at the cost of the resolution. For this reason, the elements of the convex array are arranged in a curved fashion along the azimuthal direction. The method of acquiring an image using a convex array is the same as a linear array but the ultrasound image of the convex array has a fan shape. In the case of a target object being behind obstacles it is difficult to obtain an ultrasound image using the linear array or the convex array. For this case, a phased array can be used by steering the ultrasound beams at oblique angles. Ultrasound images of a phased array have a circular cone shape.

Commonly used ultrasound data acquisition for medical applications includes focused beams, divergent beams, and planewave beams [5]. Single element transmission is seldom used in medical ultrasound imaging because it is time consuming for data collection and poor in signal to noise ratio. In ultrasound data acquisition using focused beams the time delay of each transmitter is electronically controlled in such a way that, at the focal point of a beam which is in front of the transducer and inside the image domain, transmitters employed by this beam emit sound waves that arrive at the focal point at the same time. The in-sonification at the focal point is very strong and rapidly dies down away from the focal point. In ultrasound data acquisition using divergent beams the time advance of each transmitter is electronically controlled in such a way that, at the focal point which is behind the transducer and outside the image domain, transmitters employed by this beam virtually emit sound waves from the focal point at the same time. The in-sonification in the image domain is very weak and divergent out. In ultrasound data acquisition using planewave beams the time advance of each transmitter is similar to that of a divergent beam except the virtual focal point is far away behind the transducer. All transmitters participate in the excitation of each planewave beam. The in-sonification of a planewave beam in the image domain is weak and uniform. Most commercial ultrasound scanners employ focused beams in data acquisition because the signal to noise ratio is much higher in the final image, thanks to the focusing ability. The downside of focused beams is much elevated mechanical pressure and thermal heating at the focal point, as well as much reduced frame rate compared to the planewave modality.

The present invention relates to acquisition and processing of ultrasound data for medical applications. In particular, the invention addresses two competing needs in medical diagnostic imaging: (1) overcome of noises and tissue absorption of acoustic energies in ultrasound data acquisition (2) safety concerns in terms of mechanical indices (MI) and thermal indices (TI) at focal points [1, 3]. Disclosed herein is a system and method of acquiring focusing beam ultrasound data and generating high quality images. Ultrasound scanners using focusing beams will produce similar results as ultrasound scanners using focused beams. However, at the same in-sonification energy level, ultrasound scanners using focusing beams will have much smaller mechanical indices (MI) and thermal indices (TI). Both indices are important considerations for diagnostic ultrasound imaging of infants and fetus, especially over a long period of examination time or in 3D applications. FDA imposes strict limits on both indices for all commercial ultrasound scanners. Our invention enables commercial ultrasound scanners to increase their acoustic energy levels without the corresponding increases of their mechanical indices and thermal indices. This is important for infant and fetus applications as well as imaging deep tissues in abdominal, cardiovascular, and lung ultrasound applications.

### SUMMARY OF THE INVENTION

In one embodiment, the present application discloses a method of acquiring ultrasound radio-frequency (RF) data using focusing beams. The method includes: providing an ultrasound transducer, the ultrasound transducer including a plurality of elements acting as both transmitters and receivers; transmitting sound waves from the transmitters of the ultrasound transducer within a transmit aperture with transmitter time delays being programed in such a way that the sound waves arrive at a focal point at different times but within predetermined time differences, the predetermined time differences being (i) small enough such that the in-sonification at the focal point is strong to overcome noises and attenuation of echo signals caused by tissue absorption, and (ii) yet large enough to avoid a constructive interference of the sound waves at the focal point; and receiving the sound waves using the receivers of the ultrasound transducer. The sound waves are focusing beams.

In another embodiment, the method further includes providing an another ultrasound transducer including a plurality of elements acting as receivers; and receiving the sound waves using the receivers of the another ultrasound transducer.

In another embodiment, the ultrasound transducer is a linear transducer, a curved transducer, or a matrix array transducer.

In another embodiment, the receivers of the ultrasound transducer are turned on after the transmitters of the ultrasound transducer are turned off, with or without any time delay.

In another embodiment, the receivers of the another ultrasound transducer are turned on regardless of the transmitters of the ultrasound transducer are on or off.

In another embodiment, the predetermined time difference for a given transmitter is calculated by Equations (6a) and (6b):

$$\begin{matrix}f ( \vert {x}_{s} - {x}_{c} \vert ) = \frac{\vert {x}_{s} - {x}_{c} \vert}{c} & g ( \frac{{( {x}_{s} - {x}_{c} )}^{2}}{Δ {x}^{2}} )\end{matrix} \qquad ( 6 a )$$

$$and$$

$$g ( x ) = γ sin ( α \frac{2 π}{{N}_{T}} x ) , \qquad ( 6 b )$$

where Δx is a pitch size of the ultrasound transducer, N_{T} is a number of the transmitters of the ultrasound transducer within a transmit aperture, γ is a magnitude of an oscillation of the transmitter time delays and a determines a period of the oscillation.

In another embodiment, the present application includes a method of beamforming ultrasound radio-frequency (RF) data acquired using focusing beams. The method includes: acquiring focusing beam ultrasound data using an ultrasound transducer, the ultrasound transducer including a plurality of elements acting as both transmitters and receivers; taking an input data trace of the focusing beam ultrasound data at a certain receiver location; spraying a sample of the input data trace along an impulse response curve; computing image attributes; accumulating the image contributions; repeating the above four steps for all samples of all input data traces of all focusing beams at all receiver locations; performing coherent compounding to obtain a final image; and displaying the final image. The ultrasound RF data are transmitted from the transmitters of the ultrasound transducer within a transmit aperture with transmitter time delays of focusing beams.

In another embodiment, the method further includes performing frequency filtering to protect the focusing beam ultrasound data against aliasing or wavelet distortion.

In another embodiment, the attributes include transmitter-receiver offsets of the ultrasound transducer and reflection angles at an image point.

In another embodiment, accumulating image contributions forms partial image volumes for common image point gather generation.

In another embodiment, the method further includes performing amplitude weighting for true reflection amplitude preservation.

In another embodiment, the method further includes performing post processing of raw images.

In another embodiment, displaying the final image includes transmitting the final image to a remote device for display.

In another embodiment, the ultrasound transducer is a linear transducer, a curved transducer, or a matrix array transducer.

In another embodiment, the partial image volume is either 2D or 3D.

In another embodiment, the impulse response curve is defined by the following set of formulas:

$$t ( {x}_{r} , x , z ) + t ( {x}_{s} , x , z ) = t + Δ {t}_{B} ( {x}_{s} )$$

$$\frac{\partial t ( {x}_{s} , x , z )}{\partial {x}_{s}} = \frac{\partial Δ {t}_{B} ( {x}_{s} )}{\partial {x}_{s}}$$

$$Δ {t}_{B} ( {x}_{s} ) = \frac{\sqrt{{( {x}_{s} - {x}_{F} )}^{2} + {( {z}_{s} - {z}_{F} )}^{2}}}{C} - \frac{\sqrt{{( {x}_{c} - {x}_{F} )}^{2} + {( {z}_{c} - {z}_{F} )}^{2}}}{C} + f ( \vert {x}_{s} - {x}_{c} \vert )$$

where t is a recording time, Δt_{B} is a transmitter time delay, C is a sound speed used in setting the transmitter delay, (xₛ, zₛ) is a position of the transmitters, (xᵣ, zᵣ) is a position of the receivers, (x_{F}, z_{F}) is a focal point of the focusing beam ultrasound data, (x_{c}, Z_{c}) is a center of the focusing beam ultrasound data, and (x, z) is a coordinate of an image point. In the formulas,

$$f ( \vert {x}_{s} - {x}_{c} \vert ) = \frac{\vert {x}_{s} - {x}_{c} \vert}{C} g ( \frac{{( {x}_{s} - {x}_{c} )}^{2}}{Δ {x}^{2}} )$$

$$and$$

$$g ( x ) = γsin ( α \frac{2 π}{{N}_{T}} x )$$

where Δx is a pitch size of the ultrasound transducer, N_{T} is a number of the transmitters of the ultrasound transducer within a transmit aperture, γ is a magnitude of an oscillation of the transmitter time delays and α determines a period of the oscillation.

In another embodiment, the present application provides a system for acquiring and processing ultrasound radio-frequency (RF) data using focusing beams. The system includes: an ultrasound transducer, the ultrasound transducer including a plurality of elements; a transmission and reception device; a display device; a keyboard; a pointing device; and a processing unit that contains a CPU (central processing unit) and a GPU (graphic processing unit). The CPU and the GPU are adapted to: acquire, via the ultrasound transducer and the transmission and reception device, raw RF data using focusing beams; process and send the raw RF data to CPU memories or GPU memories; beamform the raw RF data on the CPU, the GPU, or both to obtain an ultrasound image; process and send the ultrasound image to the display device; display, via the display device, the ultrasound image; and repeat the above steps for a next frame.

In another embodiment, the display device is connected to the processing unit remotely, via internet connection, wireless connection, or satellite connection.

In another embodiment, the ultrasound transducer is a linear transducer, a curved transducer, or a matrix array transducer.

In another embodiment, the keyboard is a wireless keyboard or a software keyboard installed on the processing unit.

In another embodiment, the transmission and reception device is programmed to transmit and receive various types of focusing beam.

In another embodiment, the pointing device is a touch screen.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 shows ultrasound data acquisition using a focusing beam. A linear array transducer with 128 elements is used in this illustration. The element 190 1 and #128 are fired first, followed by 190 2 and #127, . . . , and elements #63 and #64 are fired last. The Tx delays of all elements are designed in such a way that sound waves will focus towards the focal zone, but not into a point. The beam width at the focal point is not equal to zero, even at a very high frequency. The dash lines depict wavefronts of a focused beam. The (thick) solid lines are wavefronts of a focusing beam.

FIG. 2 is an illustration of transmission patterns for ultrasound data acquisition using focusing beams for a 128-element linear array transducer. The transducer is placed vertically. Three focusing beams are shown. The bottom beam has half of its transmitters and receivers within the aperture. The middle beam is a full beam, symmetrical with respect to the beam center. The top beam also has half of its transmitters and receivers within the aperture. The initial delay line is defined by the time difference between a path from the focal point to the center of the transducer and another path from the focal point to the center of a beam. The Tx delay is the time difference between two excitation instances: one is the excitation time of a transmitter and the other one is the excitation time of a virtual transmitter at the beam center location. The dash lines are corresponding Tx delays of focused beams. T0 is the time ADC recording is activated. t0 is the time the first transmitter is activated for a given beam. T0 can be less than, equal to, or great than t0.

FIG. 3 is an illustration of reception patterns for focusing beam ultrasound data acquisition with a 128-element linear array transducer placed vertically. Three focusing beams are shown. The bottom beam has half of its transmitters and receivers within the aperture. The middle beam is a full beam, symmetrical with respect to the beam center. The top beam also has half of its transmitters and receivers within the aperture. Receiving elements are turned on after a fixed time delay from the activation of the corresponding transmitting elements.

FIG. 4 shows the formation of an envelope of a set of ellipses: each ellipse is an impulse response curve in the image domain of a data sample for a single transmitter and receiver pair. The beam impulse response curve (thick line) represents a contribution path in the image space of an input RF data sample at a single receiver for all transmitters within a transmit aperture.

FIG. 5 is a workflow diagram of the beamformer of ultrasound data acquired using focusing beams: each trace of a focusing beam is beamformed by spraying all data samples onto their impulse response curves, contributing to partial image volumes in accordance with the values of an attribute associated with each point on the impulse response curves. The partial image volumes are sorted into common image point gathers. Coherent compounding is used to sum the common image point gathers to form the final image.

FIG. 6 shows a phantom model for a simulation: white dots are point scatters and white lines are continuous reflectors.

FIG. 7 shows a comparison of raw data of a focusing beam (left) and a focused beam (right) at the same location with the same settings: The focusing beam on the left has more uniform in-sonification than the focused beam on the right. The horizontal axis is receiver coordinate, and the vertical axis is time. Each display is individually normalized.

FIG. 8 shows a comparison of an image using focusing beams (left) and the same image using focused beams (right): All displays are shown in 60 dB.

FIG. 9 shows an image of focusing beam data (left) and another image with 30% random noises added to the data (right). Noise speckles are visible on the right. All displays are shown in 60 dB.

FIG. 10 shows the system architecture of a new ultrasound system according to the present invention. Focusing beams are transmitted into human tissues. Reflection echoes are received, amplified, anti-aliasing filtered, and converted into digital signals in the reception stage. Immediately after acquiring a focusing beam, the data are feed into a special beamformer to generate a partial image. Partial image memory is used to store the partial images. Upon completion of a frame the data in partial image memory are sorted into common image point gathers. The gathers are processed further and coherently compounded to form a complete image. The image is then sent to a post processor for signal enhancement, envelope computation, and logarithm conversion prior to final display and delivery.

FIG. 11 is a schematic representation of the focusing beam imaging architecture of one embodiment of the present invention.

### DETAILED DESCRIPTION OF THE ILLUSTRATED EMBODIMENTS

Reference will now be made in detail to embodiments of the present invention, example of which is illustrated in the accompanying drawings.

The present invention proposes a novel design for acquiring ultrasound data using a linear, curved, phased, or matrix array transducer. In this design the transmitter time delays within a transmit aperture are programed in such a way that sound waves from transmitters on both edges of the aperture arrive at the focal point at different times from sound waves from transmitters near the center of the aperture. The time differences are small enough such that the in-sonification at the focal point is strong to overcome noises and attenuation of echo signals caused by tissue absorption. And yet the time differences are large enough to avoid a constructive interference of sound waves at the focal point. The beamforming steps include the following: (i) take one input trace from a focusing beam; (ii) optionally perform frequency filtering to protect the data against aliasing or excessive wavelet distortion; (iii) spray data samples on the input trace along their impulse response curves calculated using equations disclosed in this invention; (iv) accumulate contributions at each output location, optionally form partial image volumes for generation of common image point gathers; (v) repeat steps (i)-(iv) for all input traces in all focusing beams; (vi) perform post processing and coherent compounding to obtain the final image.

Compared to ultrasound data acquisition using conventional focused beams, our focusing beam design can achieve comparable image quality and resolution without elevated concentration of acoustic energies at focal points, significantly reducing safety concerns such as excessive mechanical pressure and thermal heating exerted on tissues under examination. Ultrasound scanners configured with focusing beam data acquisition and processing are particularly suitable for infants and fetus examinations as well as imaging deep tissues in abdominal, vascular, cardiac, and lung applications.

### Technical Description

Focused ultrasound beams are widely used in commercial B-mode diagnostic imaging of tissues and organs [1-3]. Less common are divergent ultrasound beams and planewave ultrasound beams. We propose a new ultrasound data acquisition method that can achieve similar results as the focused beam data acquisition method, without a complete focusing of acoustic energies. Our method can significantly reduce acoustic energy concentration at a focal point. The added benefits are: (1) much smaller mechanical indices (MI) and thermal indices (TI) for a similar level of in-sonification energy, or (2) a higher level of in-sonification energy in order to achieve enhanced signal to noise ratio without appreciable increases in mechanical indices (MI) and thermal indices (TI).

### Part I: Focusing Beam Data Acquisition

### 1.1 Definition of Focusing Beams

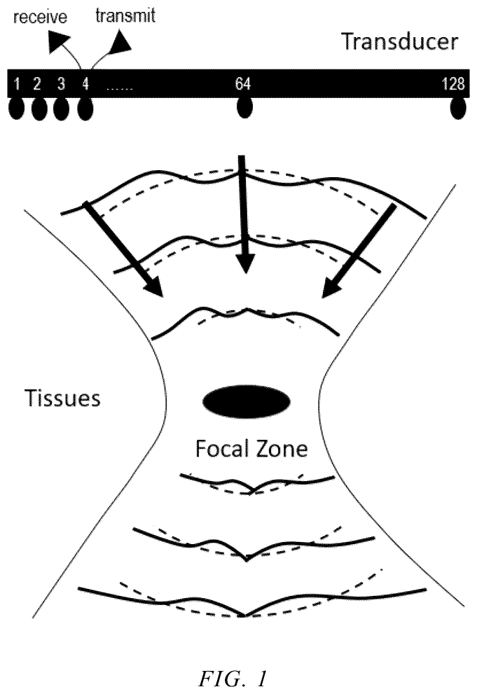

A focusing beam ultrasound dataset is collected with a modified design of the transmission pattern of a focused beam, using a transducer whose elements are arranged in a linear, curved, phased, or matrix array. In this new design of transmission pattern, the formula for Tx delay of a transmitter, in addition to the standard term of a focused beam, contains another term that is a function of distance between the element and the beam center. The additional term is chosen in such a way that sound waves from all transmitters will focus towards the focal point but not collapse into a point (FIG. 1). The effective beam width at the focal point is finite, even at a very high frequency. This beam is called a focusing beam, in contrast to a focused beam. Acoustic energies of a focusing beam will concentrate within a zone, called the focal zone.

### 1.2 Focusing Beam Transmission Design

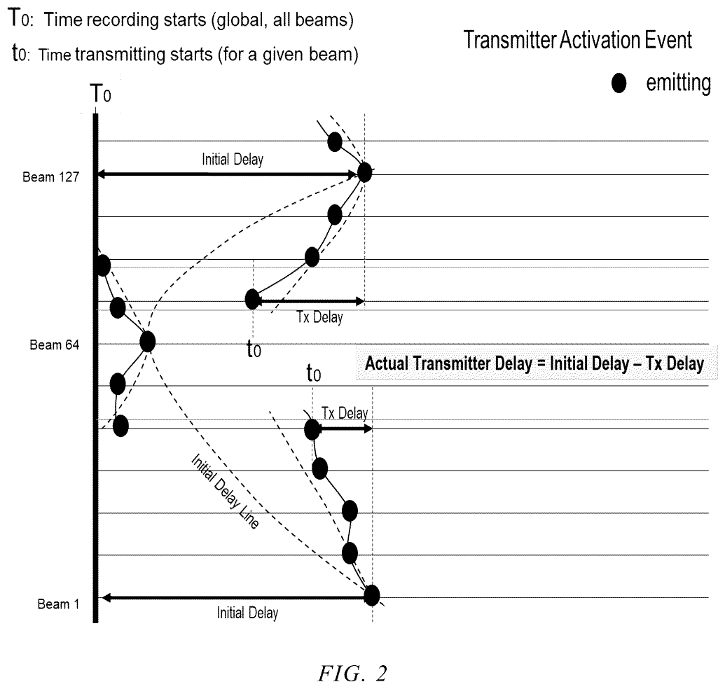

FIG. 2 shows an illustration of the transmission design for acquiring a focusing beam ultrasound dataset. The horizontal axis is lapsing time and the vertical axis is element position. We use a linear array of 128 elements as an example. The design equally applies to other array configurations, such as linear arrays with more than or less than 128 elements, curved arrays with arbitrary number of elements, phased arrays with arbitrary number of elements, or matrix arrays with arbitrary number of elements. In FIG. 2 the transducer is placed vertically at the left. Acoustic waves propagate from left to right into human tissues. The initial delay of a focusing beam is the same as a focused beam. The initial delay is the time difference between a travel path from the focal point to the center of the transducer and another travel path from the focal point to the center of this beam. However, the Tx delay of the focusing beam (solid line with dots) is different from the Tx delay of the focused beam (dash line), with a perturbation component added to the delay calculation. The total delay for a transmitter is the initial delay minus the Tx delay.

### 1.3 Focusing Beam Reception Design

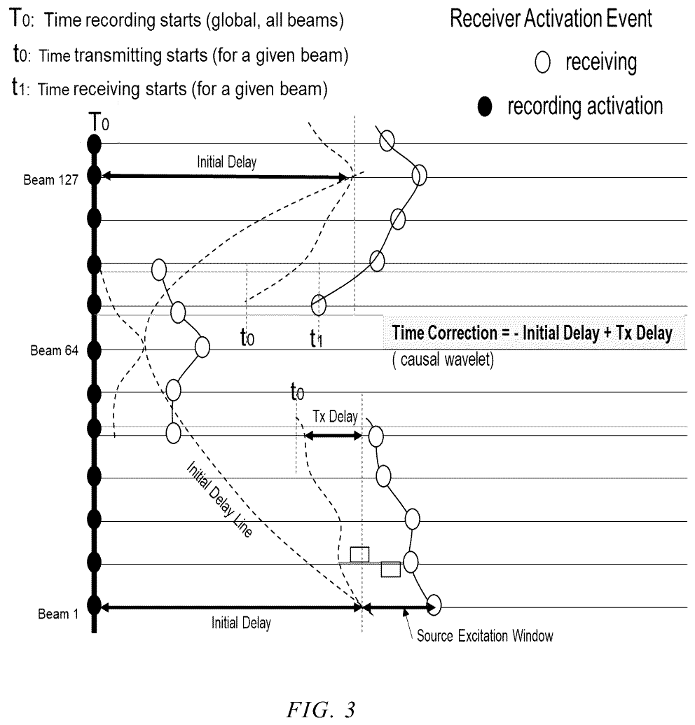

FIG. 3 shows the reception design for acquisition of ultrasound data using focusing beams. The horizontal axis is lapsing time and the vertical axis is element position. We use a linear array of 128 elements as an illustration. Other array configurations work equally well. The receivers are activated after a fixed time delay from activations of the corresponding transmitters. The fixed time delay is also called a source excitation window. In this design the ADC electronics is turned on at time TO so that we can record source signatures when some transmitters are still in activation. One can also activate the ADC electronics at some other time if recording of source signatures is not desired. In FIG. 3, time to is the start of the first transmission and time tl is the start of the first reception for each beam.

### Part II: Focusing Beam Data Processing

Traditional beamforming of ultrasound data utilizes dynamic focusing method or pixel-based beamformers for focused beams, divergent beams, or planewaves [7-12]. There is no known method existed for focusing beams. In this section we disclose a method for performing beamforming of ultrasound data that are acquired using focusing beams.

### 2.1 Beamforming Formulation of Focusing Beam Data

An input data sample at time t and at receiver location xr can be originated from a scatter at an unknown position (x, z) illuminated by an incident wave from a transmitter at location xs. The travel time satisfies the following equation:

$$t ( {x}_{r} , x , z ) + t ( {x}_{s} , x , z ) = t + Δ {t}_{B} ( {x}_{s} ) \qquad ( 1 )$$

where/is the observed time of a reflection signal at the receiver xr for a given beam. Δt_{B} is a transmitter time delay for this beam at location xₛ. (x, z) is the image (or scatter) position. t (xᵣ, x, z) is the travel time from xr to (x, z), and t (xₛ, x, z) is the travel time from xₛ to (x, z).

The above equation defines an ellipse in the image domain, which is sometime

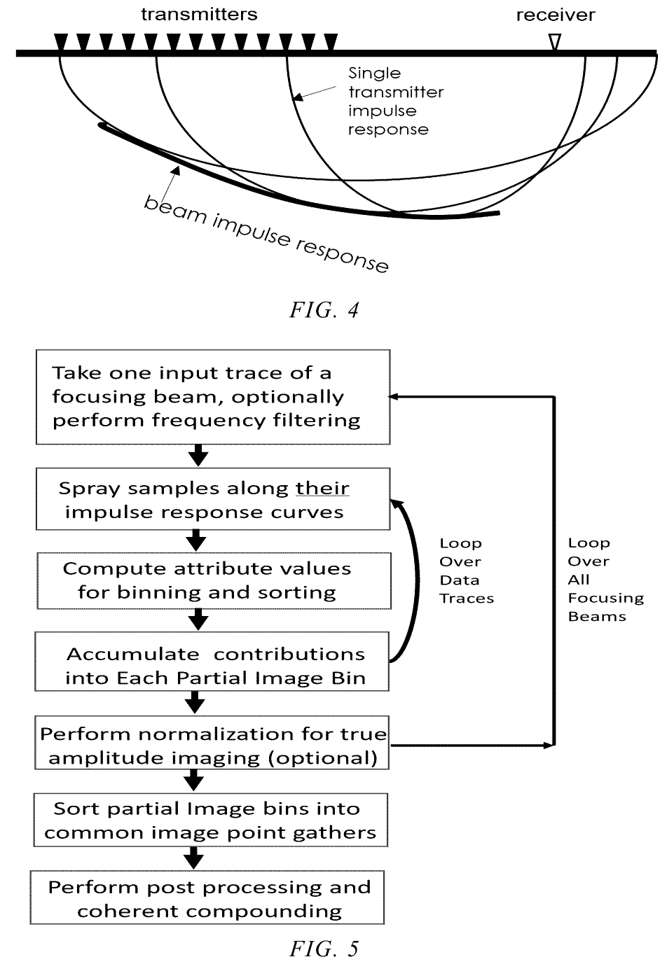

called an impulse response for a transmitter and a receiver [15]. As the transmitter position xs moves away from the beam center location xc the transmitter time delay At increases in a focusing beam. That is, as xs changes, the ellipse in equation (1) changes in both position and radius. The envelope of all the ellipses forms an impulse response curve for an input data sample of the focusing beam (FIG. 4). Please recall the input data sample is collected when many transmitters are emitting simultaneously with certain predefined time delays. The impulse response curve represents all possible spatial locations where one sample of one input beam data contributes to the image formation. The final image is the summation of all impulse response curves for all data samples in all focusing beams. This is the key concept of our beamforming method.

FIG. 4 illustrates the formation of a focusing beam impulse response (thick curve) from a collection of single transmitter impulse response curves for a given receiver. Each single transmitter impulse response curve is a trajectory in image domain on which Equation (1) is satisfied. The envelope of these single transmitter impulse response curves is a trajectory in image domain where a data sample in a focusing beam effectively contributes to. This concept applies to all types of ultrasound beam data: focusing beam, focused beam, divergent beam, planewave beam, and any other beam types.

The key to our method is to find the envelope of all the ellipses as the transmitter position changes (while holding all other geometry fixed). We define the family of curves for all the single transmitter ellipses as:

$$f ( x , z ; {x}_{s} ) = t ( {x}_{r} , x , z ) + t ( {x}_{s} , x , z ) - t - Δ {t}_{B} ( {x}_{s} ) \qquad ( 2 )$$

Its envelope, by definition, is given by:

$$f ( x , z ; {x}_{s} ) = 0 \qquad ( 3 a )$$

$$\frac{\partial f}{\partial {x}_{s}} ( x , z ; {x}_{s} ) = 0 \qquad ( 3 b )$$

The solution for (x, z) is then given by:

$$t ( {x}_{r} , x , z ) + t ( {x}_{s} , x , z ) = t + Δ {t}_{B} ( {x}_{s} ) \qquad ( 4 a )$$

$$\frac{\partial t ( {x}_{s} , x , z )}{\partial {x}_{s}} = \frac{\partial Δ {t}_{B} ( {x}_{s} )}{\partial {x}_{s}} \qquad ( 4 b )$$

Equation (4) gives a general formula for construction of an impulse response curve for one data sample of an ultrasound beam, including a focusing beam. The only requirement is that the transmitter delay function Δt_{B} (xₛ) be differentiable. We will use focusing beam data as an example, but our method equally applies to other types of ultrasound beam data.

For data acquisition using a focused beam the transmitter delay (Tx delay) is given by:

$$Δ {t}_{B} ( {x}_{s} ) = \frac{\sqrt{{( {x}_{s} - {x}_{F} )}^{2} + {( {z}_{s} - {z}_{F} )}^{2}}}{C} - \frac{\sqrt{{( {x}_{c} - {x}_{F} )}^{2} + {( {z}_{c} - {z}_{F} )}^{2}}}{C} \qquad ( 5 )$$

where C is a sound speed used in setting the transmitter delay, which may be different from the sound speed used in beamforming. (xₛ, zₛ) is the position of the transmitter. (x_{F}, z_{F}) is the focal point of this beam. The center of the beam is assumed to be at (x_{c}, z_{c}). The above equation is well known in literature [1].

For data acquisition using a focusing beam we add an oscillating component to the transmitter delay function (Tx delay) in equation (5). That is:

$$Δ {t}_{B} ( {x}_{s} ) = \frac{\sqrt{{( {x}_{s} - {x}_{F} )}^{2} + {( {z}_{s} - {z}_{F} )}^{2}}}{C} - \frac{\sqrt{{( {x}_{c} - {x}_{F} )}^{2} + {( {z}_{c} - {z}_{F} )}^{2}}}{C} + f ( \vert {x}_{s} - {x}_{c} \vert )$$

$$where :$$

$$f ( \vert {x}_{s} - {x}_{c} \vert ) = \frac{\vert {x}_{s} - {x}_{c} \vert}{C} g ( \frac{{( {x}_{s} - {x}_{c} )}^{2}}{Δ {x}^{2}} ) \qquad ( 6 a )$$

$$and$$

$$g ( x ) = γsin ( α \frac{2 π}{{N}_{T}} x ) \qquad ( 6 b )$$

are chosen to avoid discontinuity in derivative of Δt_{B} (xₛ) at xₛ=x_{c} and at the same time to approximately maintain the linearity of the function f ( ) with respect to distance from the probe center. Here Δx is the pitch size of the transducer. N_{T} is the number of elements within the transmit aperture (i.e., the number of elements used for a transmission). In equation (6b) the parameter y controls the magnitude of the oscillation of the total Tx delay and the parameter a controls the period of the oscillation. In the example below we set γ=0.25 and

$$α = \frac{2 m + 1}{{N}_{T}} ,$$

where m is an integer. Here m is the number of oscillations of the function g (x) within half distance of the transmit aperture. g (x) is not oscillating if m=0; it oscillates once if m=1; it oscillates twice if m 32 2, and so on.

It is important to point out that other functional forms can be constructed to replace equations (6a) and (6b). The concept remains the same as disclosed in this publication.

### 2.2 Relative Reduction of Mechanical Index (MI) and Thermal Index (TI)

The Mechanical Index (MI) is a quantity related to the potential for damage based on mechanical effects during a diagnostic ultrasound examination. It is defined as the ratio of peak value of rarefactional pressure (in MPa) at focal point by the square root of peak frequency (in MHz). Values of MI in diagnostic imaging generally range from 0.04-1.7 [1]. When we compare the MI of a focused ultrasound beam with the MI of a focusing ultrasound beam, all we need to know is the peak pressure difference of the two beams at the focal point (x_{F}, z_{F}) since the same probe setting is used for both beams.

The peak pressure of a focused beam is an amplified version of the peak pressure of a single transmitter directly above the focal point. The pressure amplification coefficient of a focused beam at the focal point (x_{F}, z_{F}) is given by:

$${PA}_{Focused Beam} = {\sum }_{n = - \frac{{N}_{T}}{2}}^{n = + \frac{{N}_{T}}{2}} \frac{1}{\sqrt{1 + {τ}^{2} {n}^{2}}} \qquad ( 7 )$$

The pressure amplification coefficient of a focusing beam at the same focal point is given by:

$${PA}_{Focusing Beam} = {\sum }_{n = - \frac{{N}_{T}}{2}}^{n = + \frac{{N}_{T}}{2}} \frac{{e}^{i γτ {k}_{c} {Z}_{F} \vert n \vert sin ( 2 π \frac{( 2 m + 1 ) {n}^{2}}{{N}_{T}^{2}} )}}{\sqrt{1 + {τ}^{2} {n}^{2}}} \qquad ( 8 )$$

where N_{T} is the number of elements within the transmit aperture,

$$τ = \frac{Δ x}{{Z}_{F}}$$

is the ratio of the element pitch and the focal depth,

$${k}_{c} = \frac{2 π {f}_{c}}{C}$$

is the propagation wavenumber at the peak frequency f_{c}, m, an integer, is the number of oscillations of the function g (x) within half distance of the transmit aperture.

The following table gives a comparison of the pressure amplification coefficients between a focused beam and a focusing beam. In the calculation, N_{T} =128, γ=0.25, τ32 0.01, ZF=0.03 m, f_{c}=5 MHz, and C=1540 m/s.

TABLE 1

| Pressure Amplification Coefficient | | | |
|---|---|---|---|
| (absolute value) | m = 0 | m = 2 | m = 4 |
| Focused Beam (equation (7)) | 121.40 | 121.40 | 121.40 |
| Focusing Beam (equation (8)) | 20.43 | 19.64 | 7.24 |

Pressure amplification coefficients of a focused beam (top row) and a focusing beam (bottom row) at the focal point.

As one can see in Table 1,for a focused beam, the pressure amplification coefficient at the focal point is 121.40 for a 128-element transmit aperture. The slight difference is attributed to differences in geometrical spreading of energy from transmitters away from the center. However, for a focusing beam, the pressure amplification coefficient at the focal point is only 19.64 for the same 128-element transmit aperture when m=2 is chosen, which is almost 84% smaller than that of a focused beam. As a result, the mechanical index of a focusing beam is much smaller than the mechanical index of a focused beam for the same level of in-sonification energy.

The Thermal Index (TI) provides a measure of the potential for tissue damage by heating. Under normal exposure condition it is proportional to estimated temperature rise. It is defined as the ratio between the absorbed output power and the ultrasound power required to raise the target tissue temperature by 1 degree Celsius. The absorbed output power is proportional to the square of the peak acoustic pressure. The acoustic power amplification coefficient is defined as, at the focal point, the ratio of acoustic power of an ultrasound beam and the same acoustic power of a single transmitter directly above the focal point. Therefore, the acoustic power amplification coefficient is the squared version of the corresponding pressure amplification coefficient:

TABLE 2

| Acoustic Power Amplification | | | |
|---|---|---|---|
| Coefficient | m = 0 | m = 2 | m = 4 |
| Focused Beam | 14737.96 | 14737.96 | 14737.96 |
| Focusing Beam | 417.38 | 385.73 | 52.41 |

Acoustic power amplification coefficients of a focused beam and a focusing beam at the focal point.

As shown Table 2, for a focused beam, the acoustic power amplification coefficient at the focal point is 14737.96 for a 128-element transmit aperture. However, for a focusing beam, the same acoustic power amplification coefficient is only 385.73 when m 32 2 is chosen. The reduction in thermal index (TI) of a focusing beam is very significant compared to a focused beam.

### 2.3 Implementation Details

The recommended implementation includes the following steps:
1. Take one input data trace at a receiver xr of a focusing beam;
2. Perform necessary frequency filtering to protect the data against aliasing or wavelet distortion during beamforming, if desired;
3. Spray a data sample along its impulse response curve calculated using equations (4), (5) and (6). Also compute necessary attributes such as transmitter-receiver offsets (on the transducer), reflection angles (at image points), wavelet stretch, anti-aliasing frequencies etc.;
4.Accumulate image contributions, with options to form partial images for common image point gather generation;
5.Perform amplitude normalization for true reflection amplitude preservation, if required;
6.Repeat steps (1)-(5) for all data samples of all focusing beams at all receiver locations;
7. Perform post processing and coherent compounding to obtain the final image. The implementation method disclosed herein is robust and fast when

analytical functions exist for both travel time calculation and time delay calculation. In the case where tissue sound speed varies spatially the method still yields quality images but requires a numerical solution to equation (4).

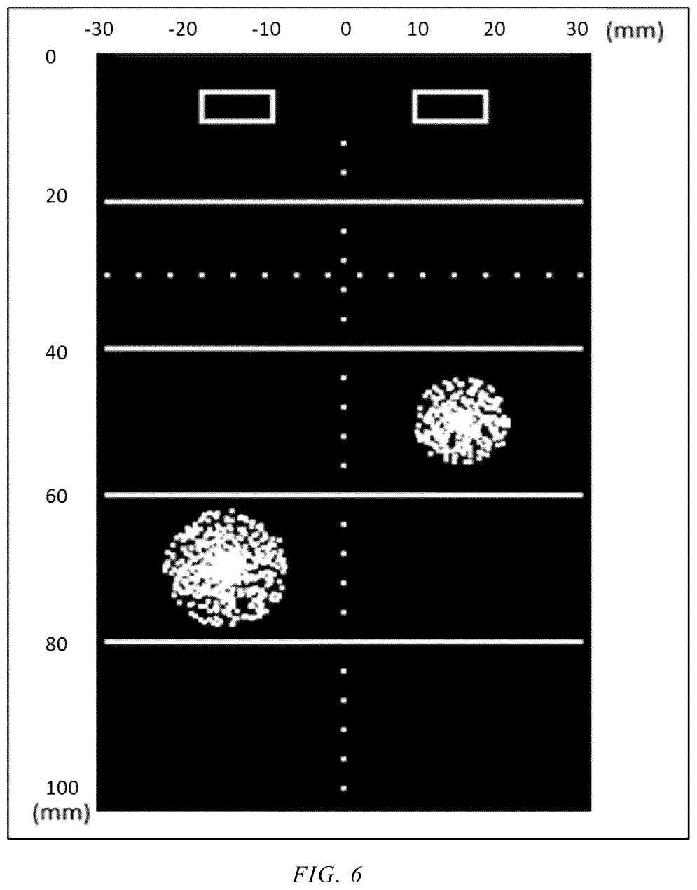

In the workflow diagram (FIG. 5), we have included the generation of partial image volumes and common image point gathers. The common image point gathers are useful for estimation of spatially varying effective sound speed values in order to produce the best ultrasound images. They are also useful for estimation of impedance and Poisson ratio properties from analysis of amplitude variation with reflection angles. They are even more useful for optimal compounding or stacking post beamforming. We have separate patent applications to cover all these aspects [13-14].

### Part III: Example

### 3.1 Echo Data Simulation

We use a modified version of Fresnel Simulator from Ultrasound Toolbox (USTB, https://www.ustb.co) for generation of numerical ultrasound beam data. The use of this simulator is subject to the citation rule. We sincerely thank the authors for making it available in the public domain [6]. The simulator is based on Fresnel approximation of diffraction of acoustic waves for rectangular transducers in a linear time invariant (LTI) system. Inputs to the simulator include a phantom model specification, a transducer specification, and a waveform specification. The phantom model used in this simulation contains (FIG. 6):
two rectangular boxes with a depth range between 7-9 mm,
4 flat continuous reflectors at 20 mm, 40 mm, 60 mm and 80 mm in depth,
A hyperechoic target with 8 mm radius at 70 mm depth and a second hyperechoic target with 6 mm radius at 50 mm depth,
A row of scatter points at 30 mm depth and a column of scatter points at the center of the model.

The probe is a linear array transducer with 192 elements (0.3 mm pitch). Each element has a width of 0.27 mm and a height of 5mm. The central frequency of the simulated echo data is 3 MHz with 80% useful bandwidth and the sampling frequency is 24 MHz. We set γ=0.25 and m=2 in equation (6) for this simulation of focusing beams.

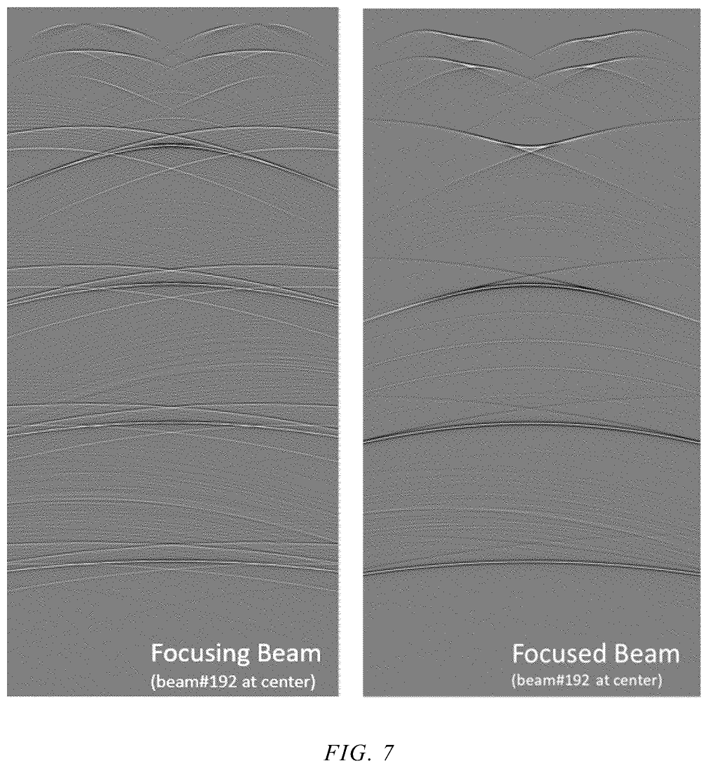

We have simulated 384 focusing beams and 384 focused beams, with beam centers equally spaced from −32.5 mm to +32.5 mm. The simulation time for a focusing beam is the same as a focused beam. FIG. 7 shows a comparison between one focusing beam (left) and one focused beam (right) at the same location. Please note that the focused beam is more concentrated in energy distribution across receive aperture than the focusing beam. The total amount of power transmitted is the same in the two beams.

### 3.2 Imaging Test

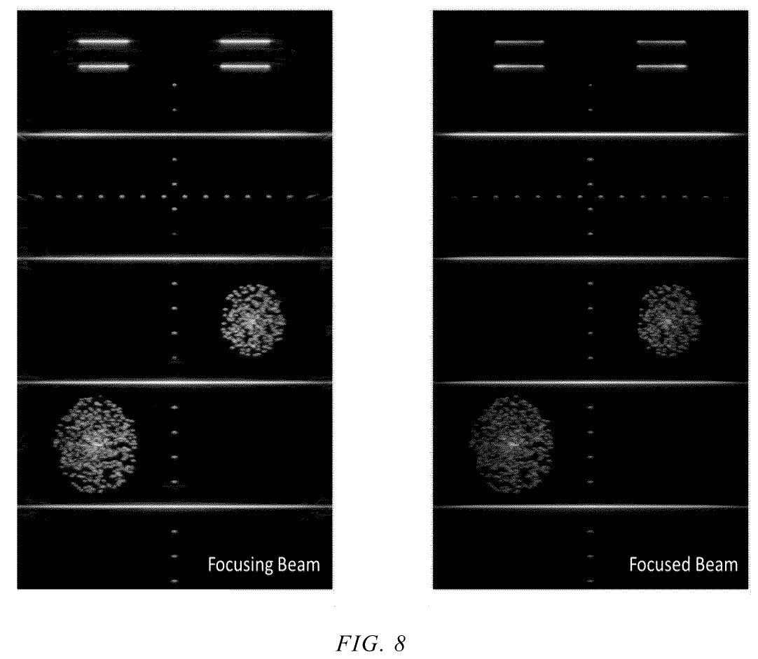

FIG. 8 shows a comparison of an image of 384 focusing beams (left) and another image of 384 focused beams (right). All parameters are the same in the two simulations. We see similar image resolution and image quality. The focusing beam image on the left is more uniform in amplitude than the focused beam image on the right. This is because the in-sonification is more uniform when focusing beams are used.

### 3.3 Signal-to-Noise Ratio Test

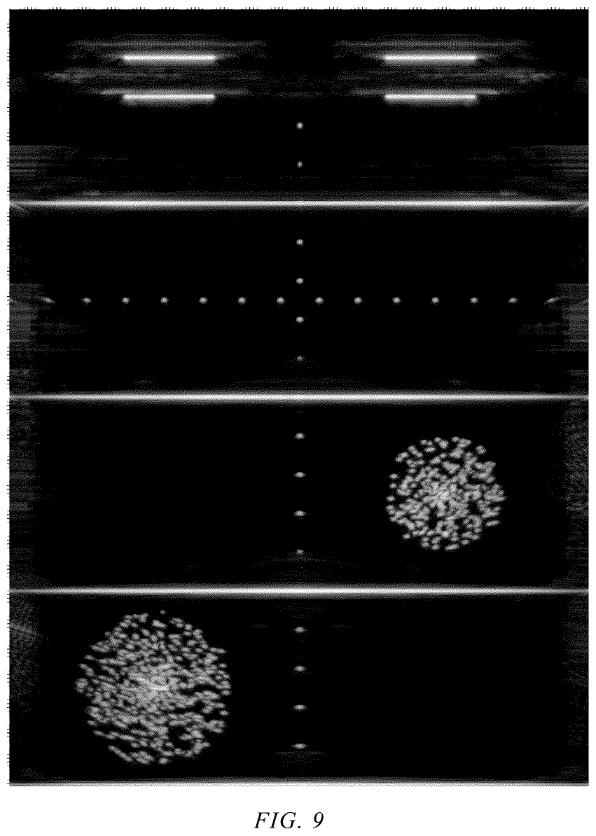

To test the impact of random data noises on image quality we add additive random noises whose maximum amplitudes are set at 30 dB while the maximum amplitudes of the original simulation data are scaled to 60 dB. FIG. 9 shows a comparison of an image of 384 focusing beams with no additive noises (left) and an image of the same 384 focusing beams with additive noises (right). We turned off operator anti-aliasing in beamforming for both. Some aliasing noises can be seen on the images. We do see some additional speckles in the image when random noises are added to the input but they almost have no impact on image quality and resolution. When we increase the noise level the speckles become stronger and plenty, especially near the two edges. We believe some speckles seen on in-vivo ultrasound images are caused by noises in data acquisition and others are real diffractors in tissues and organs. Sometimes it is difficult to distinguish between the two.

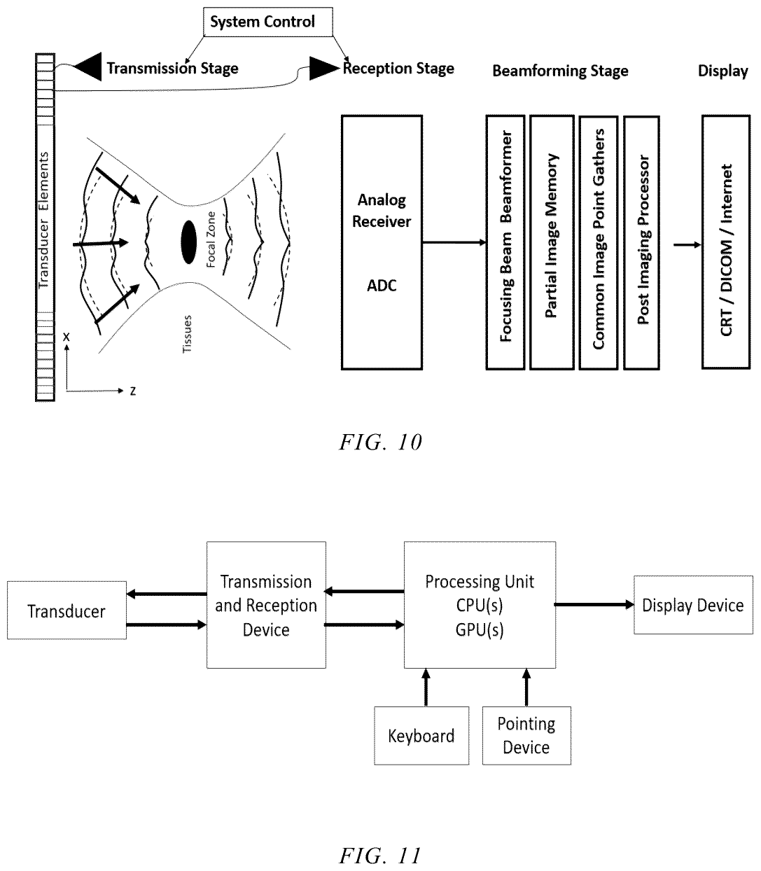

FIG. 10 shows the system architecture of a new ultrasound system according to the present invention. Focusing beams are transmitted into human tissues. Reflection echoes are received, amplified, anti-aliasing filtered, and converted into digital signals in the reception stage. Immediately after completion of acquiring a focusing beam, the data are feed into a special beamformer to generate a partial image. Partial image memory is used to store the partial images. Upon completion of a frame the image data in partial image memory are sorted into common image point gathers. The gathers are processed further and stacked to form a complete image. The image is then sent to a post processor for signal enhancement, envelope computation, and logarithm conversion prior to final display and delivery.

FIG. 11 is a schematic representation of the focusing beam imaging architecture of one embodiment of the present invention. The processing unit contains one or more CPUs and one or more GPUs. One of the CPU sends instructions to the transmission and reception device to first transmit an acoustic pulse to each element of the transducer within a transmit aperture with a time delay that is specially designed for a focusing beam, and then receive and record acoustic echoes reflected from tissue contrasts. The echo signals are sent to the processing unit for special processing and beamforming on the CPUs, GPUS, or both. The final image is displayed on a local monitor or transmit via TCP/IP to a remote display device.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

### REFERENCES

[1] Richard S. C. Cobbold (2007), Foundations of Biomedical Ultrasound, Oxford University Press, pages 431-437.
[2] O. H. Schuck (1957), Variable Focus Transducer, U.S. Pat. No. 3,090,030, May 14, 1963.
[3] P. Suetens (2009), Fundamentals of Medical Imaging. 2nd Edition, Cambridge University Press, pages 33-158.
[4] B. S. Hertzberg and W. D. Middleton (2016), Ultrasound: The Requisites, The Third Edition, Elsevier. Chapter 1, pages 3-31. Also at expertconsult.com.
[5] L. Demi (2018), Practical Guide to Ultrasound Beam Forming: Beam Pattern and Image Reconstruction Analysis, Applied Sciences, Vol 8, pages 1544-1559.
[6] A. Rodriguez-Molares, Fresnel simulator, http://www.ustb.no/examples/fresnel/[7] R. E. Daigle (2009), Ultrasound Imaging System with Pixel Oriented Processing, U.S. Pat. No. 112,095 A1, May 19, 2009.
[8] R. Zemp and M. F. Insana (2007), Imaging with Unfocused Regions of Focused Ultrasound Beams, J. Acoust. So. Amer. Vol. 121, pages 1491-1498.
[9] N. Q. Nguyen and Richard Q. Prager (2016), High-resolution Ultrasound Imaging with Unified Pixel-Based Beamforming, IEEE Transactions on Medical Imaging, Vol. 35, pages 98-108.
O. M. H. Rindal (2019), Software Beamforming in Medical Ultrasound Imaging-a Blessing and a Curse, Ph.D. Thesis, University of Oslo.
O. M. H. Rindal, A. Rodriguez-Molares, and A. Austeng (2018), A Simple, Artifact- free, Virtual Source Model, IEEE International Ultrasonics Symposium, IUS 1-4. https: ://doi.org/10.1109/ultsym.2018.8579944.
D. J. Napolitano, B. D. DeBusschere, G. W. Mclaughlin, L. Y. Mo, C. H. Chou, T. L. Ji, R. W. Steins (2011), Continuous Transmit Focusing Method and Apparatus for Ultrasound Imaging Systems, U.S. Pat. No. 8,002,705, Issued August 2011.
C. Peng, and J. Tang (2021), Acquisition and Processing of V-Wave Ultrasound Data Using a Linear or Curved Array Transducer, U.S. Patent Appl. No. 63/184,174, Filed May 4, 2021.
C. Peng, and J. Tang (2021), Imaging Tissues and Organs Behind Bones Using an Ultrasound Array Transducer, US Patent Application Ser.ubmitted.

O. Yilmaz (2011), Seismic Data Analysis: Processing, Inversion and Interpretation of Seismic Data, Society of Exploration Geophysicists.

1. A method of acquiring ultrasound radio-frequency (RF) data using focusing beams, comprising:
providing an ultrasound transducer, the ultrasound transducer including a plurality of elements acting as both transmitters and receivers;
transmitting sound waves from the transmitters of the ultrasound transducer within a transmit aperture with transmitter time delays being programed in such a way that the sound waves arrive at a focal point at different times but within predetermined time differences, the predetermined time differences being (i) small enough such that the in-sonification at the focal point is strong to overcome noises and attenuation of echo signals caused by tissue absorption, and (ii) yet large enough to avoid a constructive interference of the sound waves at the focal point; and
receiving the sound waves using the receivers of the ultrasound transducer,
wherein the sound waves are focusing beams.

2. The method of claim 1, further comprising:
providing an another ultrasound transducer including a plurality of elements acting as receivers; and
receiving the sound waves using the receivers of the another ultrasound transducer.

3. The method of claim 1, wherein the ultrasound transducer is a linear transducer, a curved transducer, or a matrix array transducer.

4. The method of claim 1, wherein the receivers of the ultrasound transducer are turned on after the transmitters of the ultrasound transducer are turned off, with or without any time delay.

5. The method of claim 2, wherein the receivers of the another ultrasound transducer are turned on regardless of the transmitters of the ultrasound transducer are on or off.

6. The method of claim 1, wherein the predetermined time difference for a given transmitter is calculated by Equations (6a) and (6b):
$$f ( \vert {x}_{s} - {x}_{c} \vert ) = \frac{\vert {x}_{s} - {x}_{c} \vert}{C} g ( \frac{{( {x}_{s} - {x}_{c} )}^{2}}{Δ {x}^{2}} ) \qquad ( 6 a )$$
$$and$$
$$g ( x ) = γsin ( α \frac{2 π}{{N}_{T}} x ) \qquad ( 6 b )$$
where Δx is a pitch size of the ultrasound transducer, N_{T} is a number of the transmitters of the ultrasound transducer within a transmit aperture, γ is a magnitude of an oscillation of the transmitter time delays and a determines a period of the oscillation.

7. A method of beamforming ultrasound radio-frequency (RF) data acquired using focusing beams, comprising:
acquiring focusing beam ultrasound data using an ultrasound transducer, the ultrasound transducer including a plurality of elements acting as both transmitters and receivers;
taking an input data trace of the focusing beam ultrasound data at a certain receiver location;
spraying a sample of the input data trace along an impulse response curve;
computing image attributes;
accumulating the image contributions;
repeating the above four steps for all samples of all input data traces of all focusing beams at all receiver locations;
performing coherent compounding to obtain a final image; and
displaying the final image,
wherein the ultrasound RF data are transmitted from the transmitters of the ultrasound transducer within a transmit aperture with transmitter time delays of focusing beams.

8. The method of claim 7, further comprising:
performing frequency filtering to protect the focusing beam ultrasound data against aliasing or wavelet distortion.

9. The method of claim 7, wherein the attributes include transmitter-receiver offsets of the ultrasound transducer and reflection angles at an image point.

10. The method of claim 7, wherein accumulating image contributions forms partial image volumes for common image point gather generation, and wherein the partial image volume is either 2D or 3D.

11. The method of claim 7, further comprising:
performing amplitude weighting for true reflection amplitude preservation.

12. The method of claim 7, further comprising:
performing post processing of raw images.

13. The method of claim 7, wherein displaying the final image includes transmitting the final image to a remote device for display.

14. The method of claim 7, wherein the ultrasound transducer is a linear transducer, a curved transducer, or a matrix array transducer.

15. (canceled)

16. The method of claim 7, wherein the impulse response curve is defined by the following set of formulas:
$$t ( {x}_{r} , x , z ) + t ( {x}_{s} , x , z ) = t + Δ {t}_{B} ( {x}_{s} )$$
$$\frac{\partial t ( {x}_{s} , x , z )}{\partial {x}_{s}} = \frac{\partial Δ {t}_{B} ( {x}_{s} )}{\partial {x}_{s}}$$
$$Δ {t}_{B} ( {x}_{s} ) = \frac{\sqrt{{( {x}_{s} - {x}_{F} )}^{2} + {( {z}_{s} - {z}_{F} )}^{2}}}{C} - \frac{\sqrt{{( {x}_{c} - {x}_{F} )}^{2} + {( {z}_{c} - {z}_{F} )}^{2}}}{C} + f ( \vert {x}_{s} - {x}_{c} \vert )$$
where t is a recording time, Δtp is a transmitter time delay, C is a sound speed used in setting the transmitter delay, (xₛ, zₛ) is a position of the transmitters, (xᵣ, zᵣ) is a position of the receivers, (x_{F}, z_{F}) is a focal point of the focusing beam ultrasound data, (x_{c}, z_{c}) is a center of the focusing beam ultrasound data, and (x, z) is a coordinate of an image point,
wherein
$$f ( \vert {x}_{s} - {x}_{c} \vert ) = \frac{\vert {x}_{s} - {x}_{c} \vert}{C} g ( \frac{{( {x}_{s} - {x}_{c} )}^{2}}{Δ {x}^{2}} )$$
$$and$$
$$g ( x ) = γsin ( α \frac{2 π}{{N}_{T}} x )$$
where Δx is a pitch size of the ultrasound transducer, N_{T} is a number of the transmitters of the ultrasound transducer within a transmit aperture, γ is a magnitude of an oscillation of the transmitter time delays and α determines a period of the oscillation.

17. A system for acquiring and processing ultrasound radio-frequency (RF) data using focusing beams, comprising:
an ultrasound transducer, the ultrasound transducer including a plurality of elements;
a transmission and reception device;
a display device;
a keyboard;
a pointing device; and
a processing unit that contains a CPU (central processing unit) and a GPU (graphic processing unit),
wherein the CPU and the GPU are adapted to:
acquire, via the ultrasound transducer and the transmission and reception device, raw RF data using focusing beams;
process and send the raw RF data to CPU memories or GPU memories;
beamform the raw RF data on the CPU, the GPU, or both to obtain an ultrasound image;
process and send the ultrasound image to the display device;
display, via the display device, the ultrasound image; and
repeat the above steps for a next frame.

18. The system of claim 17, wherein the display device is connected to the processing unit remotely, via internet connection, wireless connection, or satellite connection.

19. The system of claim 17, wherein the ultrasound transducer is a linear transducer, a curved transducer, or a matrix array transducer.

20. The system of claim 17, wherein the keyboard is a wireless keyboard or a software keyboard installed on the processing unit, and wherein the pointing device is a touch screen.

21. The system of claim 17, wherein the transmission and reception device is programmed to transmit and receive various types of focusing beam.

22. (canceled)
